(19) United States
(12) Patent Application Publication
(10) Pub. No.: US 2004/0235008 A1
(43) Pub. Date: Nov. 25, 2004

(54) Methods and compositions for profiling transcriptionally active sites of the genome

(75) Inventors: Victor I. Sementchenko, Sunnyvale, CA (US); Antonio Piccolboni, Oakland, CA (US); Philipp Kapranov, Sunnyvale, CA (US); Thomas R. Gingeras, Encinitas, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/714,253

(22) Filed: Nov. 14, 2003

Related U.S. Application Data
(63) Continuation-in-part of application No. 10/316,518, filed on Dec. 10, 2002.
(60) Provisional application No. 60/426,868, filed on Nov. 14, 2002.
(60) Provisional application No. 60/458,718, filed on Mar. 27, 2003.
(60) Provisional application No. 60/469,336, filed on May 9, 2003.
(60) Provisional application No. 60/486,376, filed on Jul. 11, 2003.

(51) Int. Cl. C12Q 1/68

(52) U.S. Cl. 435/6

(57) ABSTRACT

In some embodiments of the invention, methods are provided to profile transcriptionally active sites of the genome. The methods employ hybridization of a large number of oligonucleotide probes to nucleic acid derivatives of cytosolic RNAs derived from cell lines that respond to an exogenous stimulation.

### RELATED APPLICATIONS

This application claims priority to U.S. Provisional Application Serial Nos. 60/426,868 filed on Nov. 14, 2002; 60/458,718 filed on Mar. 27, 2003; 60/469,336 filed on May 9, 2003; and 60/486,376 filed on Jul. 11, 2003 and is a continuation-in-part of U.S. patent application Ser. No. 10/316,518 filed on Dec. 10, 2002. All these applications are incorporated herein by reference for all purposes.

### BACKGROUND OF THE INVENTION

This invention is related to biological assays, microarrays, and bioinformatics. Transcription of DNA into RNA is the basic mechanism by which cells mediate their growth, function, and metabolism. Understanding transcriptional activities, therefore, is important for uncovering the functions of the genome.

### SUMMARY OF THE INVENTION

In one aspect of the invention, methods and compositions are provided for profiling transcriptionally active sites of the genome in order to determine the biological significance of messages transcribed from unannotated portions of the genome. The methods of the invention employ, for example, oligonucleotide probes. In preferred embodiments, the oligonucleotide probes are immobilized to form high density oligonucleotide probe arrays.

In some embodiments, a method of transcriptional profiling is provided, comprising the steps of subjecting a biological sample to an exogenous stimulation, measuring transcriptional activity of the biological sample at a first differentiation stage, measuring transcriptional activity of the biological sample at a second differentiation stage and comparing the transcriptional activities from the first and second differentiated stages in at least 5 Mbases, 50 Mbases or 100 Mbases of the genome in order to obtain a transcription profile.

In preferred embodiments, a biological sample is selected in order to assess the functional significance of these transcripts. The biological sample may, for example, be an organism, a cultured organ, a tissue culture or a cell culture. In some embodiments, a cell culture is selected in order to assess the functional significance of the transcripts. In preferred embodiments, the cell culture is capable of induced differentiation. In preferred embodiments, the cell culture is a developmentally pluripotent human germ cell tumor-derived cell line such as NCCIT that responds to an exogenous morphogenic/differentiating agent such as Retinoic Acid.

Some exemplary methods of the invention have been used to interrogate the transcriptional activity of human Chromosomes 21 and 22 (Large-Scale Transcriptional Activity in Chromosomes 21 and 22, Philipp Kapranov, Simon E. Cawley, Jorg Drenkow, Stefan Bekiranov, Robert L. Strausberg, Stephen P. A. Fodor, and Thomas R. Gingeras, Science 2002 May 3;296: 916-919, which is incorporated herein by reference). The sequences of the human chromosomes 21 and 22 indicate that there are approximately 770 well-characterized and predicted genes. These genes represent only a portion of the sequence information transcribed into RNA. As shown in the cited publication (Science296:014-919, 200), empirically derived maps of the transcriptionally active areas of these chromosomes were constructed using cytosolic poly A+ RNA obtained from 11 human cell lines of diverse developmental origins. These maps were constructed using high density oligonucleotide arrays which interrogated the 35 million base pairs of non-repetitive genomic sequence, using 25 nucleotide length probes spaced on average every 30 base pairs, along these chromosomes. These results when overlaid on to the sequence annotations available for these two chromosomes reveal that as much as 9 fold more of the genomic sequences is used for transcription than envisioned by the predicted and characterized exons. These transcripts represent a hidden transcriptome not accounted for in previously annotated maps.

The above example illustrates the power of the methods of the invention in understanding the biological functions of the genome and highlights the need for large scale interrogation of transcription activity. The methods and compositions of the invention provide a powerful tool for innovative biological research, clinical diagnostics and drug development in the post genome era.

In some embodiments, the method for interrogating transcriptional activity includes the steps of obtaining a polyA+ RNA sample from a cellular compartment; hybridizing the polyA+ RNA or nucleic acids derived from the RNA with an oligonucleotide probe array, wherein the oligonucleotide probe array contains at least 10,000 oligonucelotide probes designed to be perfect match (PM) probes, each of the perfect match probes targets a different transcript sequence from a region of a genome; and determining that a genomic sequence is transcribed if the probe against the genomic sequence is hybridized with a target.

While the method of the invention may be employed for interrogating the transcriptional activities in a genomic region of any size, the method is particularly useful for interrogation a large genomic region, for example, a region of at least 20 MB, 50 MB and higher, or 25%, 50%, 100% of the DNA sequences in a chromosome. In some embodiments, the DNA sequence from an entire genome is interrogated in a set of 1, 2, 5, 10, 50, or 100 probe arrays.

The probes may target the transcript sequences from the genome at a resolution of at least 100 bps, 30 bps, 10 bps, or 1 bp.

The RNAs from different cellular compartments, such as cytosol or nuclei, may be detected using the methods of the invention.

Typically, each of the oligonucleotide probe arrays contains at least 100,000, 500,000 or 800,000 oligonucleotide probes, each targeting a transcript sequence from a different region of a genome. The oligonucleotides are immobilized at a feature (each area which is designed to contain a probe is a feature) size of smaller than 20, 15, 14, 10, 8, 5, 2, 1 microns.

In addition to perfect match (PM) probes, the oligonucleotide arrays may also contain oligonucleotides designed to be mismatch (MM) probes. Each of the mismatch probes is different from a perfect match probe in one base. In preferred embodiments, a mismatch probe is different from a perfect match probe in a middle position. Other control probes may also be included.

The perfect match probes are typically selected according to the genomic sequence, desired interrogation resolution. In preferred embodiments, repetitive sequence of the genome is filtered and not used as interrogation regions.

The transcriptional activity profiles may be obtained under different conditions such as normal vs. diseased, different physiological and pathological conditions, various chemical treatments. These profiles may be compared to reveal transcriptional activities that may be related to physiological, pathological or toxicological conditions.

The transcriptional activity profile may be used to guide the verification and isolation (cloning) of novel transcripts. These profiles may also be used to decipher the regulatory mechanisms. In addition, the transcriptional activity profiling may be employed for clinical diagnostics, toxicity testing (e.g., for drug candidates), and drug development.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1: High resolution maps of four regions within DGCR of chromosome 22 (22q11.2). For each map the contigs predicted by the DGCR array for 6 of the 11 cell lines analyzed is presented. Below the array map are cartoons derived from the Sanger hand curated map of this region or EST maps derived from dbEST. Selected regions suggested by the array map were further analyzed using RT-PCR. The sequenced products from these analyses are mapped below the Sanger and ESTs maps (A) DCGR6 gene region (GP sequence 15,833,950-15,840,390), (B) DGCR2 region(GP sequence 15,959,850-16,057,850), (C)SLC25A1 25 and 5′ flanking region (GP sequence 16,098,590-16,107,090), (D)DGCR5 exon1 region(GP sequence 15,898,300-15,905,040).

FIG. 2: Correlation of the positive probe and exon density maps (5% FP rate) for Chromosomes 21 (A) and 22(B). For each map the lowest graph depicts the positive probes density present in 57 kb bins (average genomic size for genes on chromosomes 21). Above this plot is the density of nucleotides located within exons present in each bin. The graph overlaying a cartoon of each chromosome is the local correlation coefficient of the exon density and the positive probe density calculated over a 5.7 Mb window. A correlation coefficient is not calculated in regions where the percentage of positive exon density falls below 25% over the 5.7Mb window. Thus, chromosome 21 region near the centromer that is relatively sparse in exon annotations is not analyzed for correlation with positive probe density given the relative lack of variation in the exon density in these chromosomal regions. Above the positive probe density maps are the regions selected for RT-PCR and Northern hybridization verification (downward arrows). The DGCR region of chromosome 22 is boxed on (B). High resolution maps of the DGCR is shown in FIG. 1.

FIG. 3: Northern hybridization analyses of poly A+ cytosolic RNA obtained from 7 of the 11 cell lines (1: NIH:OVCAR-3, 2: Jurkat, 3: HepG2; 4: FHs 738Lu; 5: COLO 205; 6: CCRF-CEM; 7: A-375; 8: A-375 treated with DNAse I.). The following probes were radioactively labeled and hybridized to the filters: (A) a cDNA derived from Chr22 DGCR-3-2 region (table 3, Example) and represented by bp 277304-277569 of the DGCR sequence; and cDNAs spanning entire validated regions (B) Chr22 DGCR-2-1; (C) Chr21-8 and (D) Chr22 DGCR-1-2. The films were exposed for 3 weeks.

FIG. 4: Classification of transfrags that are up or down-regulated by 2-fold or more.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays which are also described.

Nucleic acid arrays that are useful in the present invention include those that are commercially available from Affymetrix (Santa Clara, Calif.) under the brand name GeneChip®. Example arrays are shown on the website at affymetrix.com. The present invention also contemplates many uses for polymers attached to solid substrates. These uses include gene expression monitoring, profiling, library screening, genotyping and diagnostics. Gene expression monitoring and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598, which are incorporated herein by reference for all purposes.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. No. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170, which are incorporated herein by reference.

Additionally, the present invention may have preferred embodiments that include methods for providing genetic information over networks such as the Internet as shown in U.S. patent applications Ser. No. 10/063,559, 60/349,546, 60/376,003, 60/394,574, 60/403,381.

II. Glossary

The following terms are intended to have the following general meanings as used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

A nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases (see, e.g., U.S. Pat. No. 6,156, 501, incorporated herein by reference). The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. “Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents-which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids. See, e.g., U.S. Pat. No. 5,143,854.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tm) fro the specific sequence at a defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

Effective amount refers to an amount sufficient to induce a desired result.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful. See, e.g., Dong et al., Genome Research 11, 1418 (2001), in U.S. Pat. No. 6,361,947, 6,391,592, incorporated herein by reference.

A primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments represents the whole or a portion of a genome. Frequently, a genomic library is a collection of clones made from a set of randomly generated, sometimes overlapping DNA fragments representing the entire genome or a portion of the genome of an organism.

An allele refers to one specific form of a genetic sequence (such as a gene) within a cell or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

At each autosomal specific chromosomal location or “locus” an individual possesses two alleles, one inherited from the father and one from the mother. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

III. Methods for Determining Transcriptional Activity

In one aspect of the invention, methods are provided for interrogating the transcriptional activity of genome using oligonucleotide probes. As the example shows, the methods of the invention are powerful tools in uncovering the often transcription activity of a genome and provide valuable information about the functions of the genome. The methods have many practical applications in biology, medicine, environmental science, industrial biotechnology, pharmaceutical industry and many other fields.

The exemplary methods of the invention have been successfully used to uncover the hidden transcription activity of human Chromosome 21 and 22 (Large-Scale Transcriptional Activity in Chromosomes 21 and 22, Philipp Kapranov, Simon E. Cawley, Jorg Drenkow, Stefan Bekiranov, Robert L. Strausberg, Stephen P. A. Fodor, and Thomas R. Gingeras, Science 2002 May 3;296: 916-919, which is incorporated here by reference). Many of the uncovered transcripts have been verified using several different technologies including the traditional Northern blotting/hybridization and RT-PCR.

Transcriptionally active regions of the human genome have been mapped based on a combination of the alignment of cDNA sequences to genomic sequences and the interpretation of genome sequences to predict coding regions (See for example, LocusLink on the NIH website; Rubin, G. M. et al. Science 287, 2012 (2000); Caron, H., et al. Science 291, 1289 (2001); Wright, F. A. et al. Genome biology 2, 1 (2001)). In comparison with other approaches, the approach provided in this application has several advantages including: the identification of new regions of transcription not previously observed by previous experimentation or sequence analysis, the detection of RNA transcripts which have little or no coding capacity and the identification of alternative RNA isoforms of previously annotated genes.

In some embodiments, the method for interrogating transcriptional activity includes obtaining a polyA+ RNA sample from a cellular compartment; hybridizing the polyA+ RNA or nucleic acids derived from the RNA with an oligonucleotide probe array, wherein the oligonucleotide probe array contains at least 10,000, 50,000, 100,000, 500,000, or 1,000,000 perfect match (PM) probes, each of the perfect match probes targets a different transcript sequence from a region of a genome; and determining that a genomic sequence is transcribed if the probe against the genomic sequence is hybridized with a target.

In this approach, RNA samples are prepared by first separating the nuclear and cytosolic cellular compartments and then fractionating the RNA transcripts into total or poly A+ containing RNAs. Methods for separating nuclear and cytosolic cellular compartments and for isolating RNAs and poly A+ containing RNAs are well known in the art and an exemplary method is described in the example below.

By focusing on RNA sub-populations that are specifically transported to the cytoplasm and enriched for the most mature and processed forms of RNA, the methods allow for the detection and identification of rare and potentially interesting RNA transcripts that because of the effects of dilution have not been observed previously in this RNA pool. However, the methods of the invention are not limited to the use for cytosolic poly A+ RNAs. For example, in one example, polyA+ RNAs from nuclei were interrogated using high density oligonucleotide probe arrays. The transcript profile from the nuclei was compared with the profile from the cytosolic RNA to reveal interesting difference and may be related to certain biological function.

While it is possible to directly hybridize poly A+ RNA to a high density oligonucleotide probe array, it is often preferred to use derived nucleic acids instead. Derived nucleic acids are obtained using the sample RNAs as templates. Derived nucleic acids may be DNAs (such as cDNAs) or RNAs (such as cRNAs) or their analogs or mimics. Many methods may be used to make derived nucleic acids including cDNA synthesis using random primers (see the example for an exemplary protocol). cRNAs can be made using cDNA as templates in an in vitro transcription reaction. Nucleic acid amplifications, such as PCR, LCR, strand displacement amplification, in vitro transcription, etc., may be employed, for example, to increase the detection sensitivity.

It is important to note that during the process of making derived nucleic acids, certain bias towards 5′ or 3′ end sequences may occur, depending upon the methods used to make derived nucleic acids. In some embodiments, unbiased or less biased methods may be preferred. In other embodiments, methods that are biased toward the 5′ end and methods biased toward the 3′ end may be used in conjunction to interrogate both the 5′ and 3′ end of a transcript.

Typically, the nucleic acids are labeled for ease of detection. Nucleic acid labeling technology is well known in the art and is described in many of the patents/patent applications incorporated by reference above. One preferred labeling method is described in the example section below. One of skill in the art would appreciate that many embodiments of the methods of the invention are not dependent upon specific labeling methods. In fact, the methods may also be used with nucleic acid detection technology that does not employ labels.

While the method of the invention may be employed for interrogating the transcriptional activities in a genomic region of any size, the method is particularly useful for interrogation a large genomic region, for example, a region of at least 20 MB, 50 MB or higher, or 25%, 50%, 100% of the DNA sequences in a chromosome. In some embodiments, the DNA sequence from an entire genome is interrogated in a set of 1, 2, 5, 10, is 50, or 100 probe arrays.

The probes may target the transcript sequences from the genome at a resolution of at least 100 bps, 30 bps, 10 bps, or 1 bp.

Typically, each of the oligonucleotide probe arrays contains at least 100,000, 500,000, 800,000 oligonucleotide probes, each targeting a transcript sequence from a different region of a genome. The oligonucleotide probes may be 15, 20, 25, 30,35,40,45, 50, 55, or 60 bases long. They can be synthesized on a substrate, for example, using photo directed synthesis. Alternatively, they can be pre-synthesized and spotted onto a substrate to form microarrays. In preferred embodiments, however, the oligonucleotide probes are 25 mers and synthesized using photo directed synthesis. The oligonucleotides are immobilized at feature (each area which is designed to contain a probe is a feature) size of smaller than 20, 15, 14, 10, 8, 5, 2, 1 microns.

In addition to perfect match probes, the oligonucleotide arrays may also contain oligonucleotide designed to be mismatch (MM) probes. Each of the mismatch probes is different from a perfect match probe in one base. In preferred embodiments, mismatch probes is different from the perfect match probe in a middle position. Other control probes may also be included.

The perfect match probes are typically selected according to the genomic sequence, desired interrogation resolution. In preferred embodiments, repetitive sequence of the genome is filtered and not used as interrogation regions.

In another aspect of the invention, methods are provided for the determination of whether a probe pair detects an RNA target. In some embodiments, a positive detection is made using a range of threshold values for the ratio (R) of PM to MM measurements and for the difference (D) of PM-MM values. A probe pair with background-subtracted perfect match intensity PM and mismatch intensity MM is called positive if the ratio PM/MM exceeded some ratio threshold R and the difference PM-MM exceeded a difference threshold D, otherwise it is termed negative. Varying the thresholds yields different levels of sensitivity and specificity. Transcriptional Maps can be generated using R in the range 1.1 through 1.5, and D in the range 4Q through 12Q, where Q, the pixel variation within features belonging to the 2nd percentile value of probe intensities for the chip, is an estimate of noise variation.

In some embodiments, particularly in case of high resolution detection, such as the 1 bp resolution, it is desirable to increase the confidence of calls made by each probe pair by asking if neighboring probes also possessed values that exceeded R and D thresholds. By setting a minimum number of adjoining probes (minrun) and maximum gap (maxgap) between adjoining probes, maps with contiguous (contigs) runs of RNA can be built. Maps can be improved by taking into account local probe behavior in a heuristic two-step process. For example, in the first pass, runs of negative probe pairs in between positive probe pairs can be re-classified as positive if the run-length was at most maxgap bases in length. In the second pass, runs of positive probe pairs of length less than minrun bases can be reclassified as negative. The effect of the steps is to reduce the false negative and false positive rates. Exemplary values of maxgap and minrun can be 5 and 20 respectively.

Computer software and computer systems are employed to perform data analysis. The computer software may include computer software codes that perform the methods of data analysis (for example, determining whether a probe pair detects a RNA). The computer program codes are typically stored in a suitable computer readable medium such as a hard drive, a CD-ROM, a DVD-ROM, etc. Computer systems for data analysis are computer systems (including computer networks) for executing the data analysis of the invention.

In another aspect of the invention, spiked RNA transcripts may be used as a control. For example, in assays for human transcripts, bacterial RNA transcripts containing specific sequence deletions can be placed in each polyA+ RNA sample. The bacteria transcripts may be used for estimating sensitivity and false positive rate (see example below).

The transcriptional activity profiles may be obtained under different conditions such as normal vs. diseased, different physiological and pathological conditions, various chemical treatments. These profiles may be compared to reveal transcriptional activities that may be related to physiological, pathological or toxicological conditions (see, e.g., U.S. Pat. No. 6,033,860).

In one aspect of the invention, the transcriptional activity profiles may be stored in computer databases (such as a relational data base). The profiles may be searched, summarized and analyzed in various ways.

The transcriptional activity profile may be used to guide the verification and isolation (cloning) of novel transcripts. For example, if a region of the genome is detected to be transcribed, primers may be designed to perform RT-PCR to verify and isolate the transcribed sequence (see the example section for an example). The isolated cDNA may be studied for its functions.

In another aspect of the invention, the transcriptional activity profiling using the methods of the invention may be employed for clinical diagnostics. In such applications, a transcriptional activity profile obtained from a patient sample may be compared with one or more reference profiles (diseased or normal) to detect the similarity of the transcriptional activity pattern with the reference profiles. The reference profiles may be obtained by interrogating diseased and normal tissues for transcriptional activity using the methods of the invention.

Transcriptional activity profiling may be also used for in vitro toxicity testing. In such applications, a chemical compound is used to treat a cell culture. The transcriptional activity of the cells may be interrogated. The profile of the transcriptional activity may be compared with reference profiles to detect whether the compound may have toxic effort. The reference profiles may be generated by testing known toxic and nontoxic compounds for toxic and non toxic transcriptional activity profiles.

Similarly, transcriptional activity profiling may be used for testing drug candidates. In such applications, a drug candidate may be tested in cell cultures to determine whether it induces desirable transcriptional activity.

In yet another aspect of the invention, the transcriptional activity discovered using the methods of the invention may be used for designing microarrays for gene expression monitoring. For example, the transcriptional maps may be used to identify novel transcripts. Probes targeting the novel transcripts may be designed and immobilized on a substrate to form a microarray that can be used to monitor the expression of the novel transcripts.

The following examples illustrate various aspects of the invention.

IV. Example-Large Scale Transcriptional Activity of the Human Genome in Chromosomes 21 and 22

To demonstrate the power of the methods of the invention, the methods are used to develop an empirical map of the transcriptionally active regions of the human genome at the nucleotide level and relate this map to the sequence annotations derived from other approaches.

Oligonucleotide Probe Arrays

Arrays were created with oligonucleotide probes that interrogate the sequences of human chromosomes 21 and 22 in a systematic fashion using uniformly spaced probes that either interrogate every base or on average every 30 base pairs (bp). The advantages to this approach are several including: the identification of new regions of transcription not previously observed by previous experimentation or sequence analysis, the detection of RNA transcripts which have little or no coding capacity and the identification of alternative RNA isoforms of previously annotated genes.

Sample Preparation

One important aspect to this experimental effort of identifying transcriptionally active regions of chromosomes 21 and 22 has been the preparation of target cellular RNA transcripts that are to be mapped. RNA samples were prepared by first separating the nuclear and cytosolic cellular compartments and then fractionating the RNA transcripts into total or poly A+ containing RNAs. This sample preparation approach complements an unbiased strategy in searching for transcriptionally active regions of chromosomes 21 and 22 by allowing the analysis to focus on RNA sub-populations that are specifically transported to the cytoplasm and enriched for the most mature and processed forms of RNA. In turn, this allows for the detection and identification of rare and potentially interesting RNA transcripts that because of the effects of dilution have not been observed previously in this RNA pool.

Experimental Design and Error Estimates

A total of 11 different cell lines of diverse developmental origin were used to obtain the RNAs: A-375 (melanoma, ATCC no CRL-1619); CCRF-CEM (acute lymphoblastic leukemia; T lymphoblast); COLO 205 (colorectal adenocarcinoma, ATCC no. CCL-222); FHs 738Lu (normal fetal lung fibroblasts, ATCC no. HTB-157); HepG2 (hepatoblastoma, ATCC no. HB-8065); Jurkat (acute T cell leukemia); NCCIT (teratocarcinoma, ATCC no. CRL-2073); NIH:OVCAR-3 (ovarian adenocarcinoma, ATCC no. HTB-161); PC3 (prostate adenocarcinoma, ATCC no. CRL-1435); SK-N-AS (neuroblastoma, ATCC no. CRL-2137); U-87 MG (astrocytoma, ATCC no. HTB-14). Jurkat and CCRF-CEM were obtained from Dr. Jacques Corbeil, Center for AIDS Research and Veterans Medical Research Foundation, University of California San Diego.) Each cell line was prepared by separating the nucleus and cytoplasmic compartments and the RNAs present in each were fractionated to obtain the polyA+-containing subfraction. Total cytosolic RNA and its polyA⁺ fraction were prepared using RNeasy and Oligotex kits (Qiagen) following the manufacturer's instructions. mRNA was mixed with random hexamers (83.3 ng/μg of mRNA; Life Technologies) and the bacterial control transcripts (see below) and subjected to the following cycling conditions in PE GeneAmp9600 PCR System: 70° C.- 10 min and 10 min ramp to 25° C. after which the 5× Superscript II First Strand buffer (Life Technologies), DTT and four dNTPs were added to the following final concentrations of 1×, 10 mM and 0.5 mM, respectively followed by a 10 min incubation at 25° C. At this point, Superscript II RTase was added (200Units/μg of mRNA; Life Technologies) followed by a 10 min ramp to 42° C. and 60 min incubation at 42° C.

The volume of the first strand cDNA synthesis reaction was 20 μl per every 3 μg of mRNA. After inactivation of the RTase for 15 min at 70° C., the first strand cDNA was split in 20 μl aliquots and used as a template for the second strand cDNA synthesis using conditions described in the SuperScript Choice System for cDNA synthesis Manual (Life Technologies). After the second strand synthesis reaction, the mRNA template was degraded using a combination of RNAseA/T1 cocktail (Ambion) and RNAse H (Life Technologies). The second-strand synthesis reactions from each cell-line were pooled, purified using QIAquick PCR purification kit (Qiagen), ethanol-precipitated and subjected to a limited DNAse I (Epicenter Technologies) digest to generate fragments of 50-100 bp. The cDNA was labeled in 70 μl using 100 units of terminal transferase (Roche) and 71.4 μM of Biotin-N6-ddATP for 2 hrs at 37° C., after which it was directly used for hybridization in the following mixture: 30 mM MES (Sigma M-2933); 74 mM MES•Na (Sigma M-3058); 3M Tetramethylammonium chloride (Sigma T-3411); 0.1 mg/ml herring sperm DNA (Life Technologies); 0.02% Triton X-100; 1X Eukaryotic Hybridization Controls (Affymetrix), 0.05 nM control biotinylated oligos 948 or 213 (Affymetrix). Typically, 1-2 μg of double-stranded labeled cDNA was used per hybridization.

Hybridization and Detection

The oligonucleotide probe arrays (chips) for interrogating transcriptional activity were hybridized 16-18 hours at 45° C. Washing was done using the antibody amplification protocol as described in the Affymetrix Expression Analysis Technical Manual. Chips were scanned on GeneArray® scanner using the highest PMT settings and 2 μm pixel. Each sample was hybridized in triplicates.

Since the cDNAs copied from RNA of this subfraction were labeled and used as targets for the arrays, careful attention was paid to removal of possible DNA contamination in this step. Cytosolic polyA+ RNA from NCCIT and COLO 205 cell lines was treated with RNase-free DNAse I (2 Units/μg of mRNA; Roche) in presence of 10 mM Tris-acetate (pH7.5), 10 mM magnesium acetate, 50 mM potassium acetate, 1Unit/μl ANTI-RNAse (Ambion) for 1 hour at 37° C. As a control for DNAse I digest, the reaction was spiked with the control DNAs (1 ng/μg of mRNA) corresponding to the plasmids containing the following segments from each of the three bacterial controls LYS 328-1344, PHE 2016-3331, THR 247-2231 (see below for full description of these control genes). After DNAse I digest, the mRNA was purified by phenol/chloroform extraction and ethanol precipitation and used for cDNA synthesis and hybridization to the Chrom21_{—}22 and DGCR arrays as described above. The number of the probes hybridizing within the known exons and outside of annotated regions was calculated and found not to be significantly different to these from the corresponding untreated samples (data not shown). As an additional control for genomic DNA contamination, total cytosolic RNA and its polyA⁺ fraction was pre-treated with DNAse-free RNAse (Roche) prior to RT-PCR reactions.

Additionally, the separation of the RNAs present in the nucleus and cytoplasm was evaluated using commercially available high-density oligonucleotide arrays (such as the GeneChip® HG_U-95 probe array). Total RNA derived from cytosol or nuclear fractions of each cell line was converted into single-stranded cDNA using random primers, fragmented with DNAse I and end-labeled with terminal transferase as described above without the second strand cDNA synthesis . This cDNA was hybridized to GeneChip® HG_U-95A arrays in duplicate experiments. Expression of human Xist gene was monitored using probe set 38446_at, and was found to be nuclear-specific only in the female-derived cell lines. In addition, a number of cDNAs of unknown functions containing LINE, HERV and other types of repeats as well as unique regions were frequently detected in the nuclear, but not the cytosolic fraction in various cell lines.

Sets of oligonucleotide probes selected to interrogate the X-chromosome inactivation gene (Xist) present on the GeneChip® HG U_{—}95A arrays (Affymetrix) were used to test the quality of the nuclear/cytoplasmic separation techniques. Analysis of nuclear and cytoplasmic RNA fractions from Jurkat, CCRF-CEM, SK-N-AS, A375, HepG2, NCCIT and FHs 738Lu cell lines indicated that expression of the Xist gene was detected only in the nuclear RNA fraction of the female derived CCRF-CEM, SK-N-AS and A375 cell lines. Expression of this gene was not detected in the nuclear fraction of male derived cell lines nor in the cytoplamsic RNAs obtained from any of the cell lines (data not shown). A dditionally, separations of nuclear and cytoplasmic RNA compartments allowed for the enrichment of low copy number RNAs.

An increase in the detection of the expression of approximately 10-20% of total genes could be observed after RNA enrichment that accompanied nuclear and cytoplasmic fractionation.

Labeled cDNAs made from cytoplasmic polyA+ RNA fraction from 11 cell lines were hybridized to high-density oligonucleotide (25 mers) arrays made within individual synthesis features of 14×14 microns. These arrays contained approximately 800,000 interrogating probes. Using this probe density, two array designs were employed. The first array design interrogated 362,901 contiguous nucleotides of chromosome 22 using a perfect complement (PM) and mismatch (MM) complement oligonucleotide probe set for each base. This single base interrogation design (DGCR array) was used to map the RNA transcripts localized in the DiGeorge's syndrome critical region (DGCR) of chromosome 22 (22q11.2) (Driscoll, D. A., et al. Am J. Hum Genet. 50, 924 (1992); Greenberg, F., et al. Am. J. Hum. Genet. 43, 605 (1988); Cary, A. H., et al. Am. J. Hum. Genet. 51, 964 (1992)). The second array design interrogated 35 million non-repetitive base pairs of chromosomes 21 and 22 (Chrom 21_{—}22 arrays) using 1,011,768 probe pairs synthesized on a three array set. Oligonucleotide probe sequences were selected using empirically based rules developed at Affymetrix and pruned against the Unigene 95 database and chromosome 21 and 22 sequences for potential full or partial homologues. Each probe pair on the Chrom 21_{—}22 array interrogated the non-repeat genomic sequences on average by 30 bases. Repeat sequence regions of these chromosomes were identified by use of the RepeatMasker software (available on the University of Washington's website).

Data Analysis

Determination of whether a probe pair detected an RNA target was made using a range of threshold values for the ratio (R) of PM to MM measurements and for the difference (D) of PM-MM values. A probe pair with background-subtracted perfect match intensity PM and mismatch intensity MM is called positive if the ratio PM/MM exceeded some ratio threshold R and the difference PM-MM exceeded a difference threshold D, otherwise it is termed negative. Varying the thresholds yields different levels of sensitivity and specificity. Maps were generated using R in the range 1.1 through 1.5, and D in the range 4Q through 12Q, where Q, the pixel variation within features belonging to the 2nd percentile value of probe intensities for the chip, is an estimate of noise variation. Because of the overlap of interrogating probes used in the design of the DGCR array, it was possible to increase the confidence of calls made by each probe pair by asking if neighboring probes also possessed values that exceeded R and D thresholds. By setting a minimum number of adjoining probes (minrun) and maximum gap (maxgap) between adjoining probes, it was possible to build maps with contiguous (contigs) runs of RNA. Maps were improved by taking into account local probe behavior in a heuristic two-step process. In the first pass, runs of negative probe pairs in between positive probe pairs were re-classified as positive if the run-length was at most maxgap bases in length. In the second pass, runs of positive probe pairs of length less than minrun bases were reclassified as negative. The effect of the steps is to reduce the false negative and false positive rates. The values of maxgap and minrun used were 5 and 20 respectively.

Contigs for the chrom 21_{—}22 array data were not constructed because of the distance between the probes used in this design. By fixing the R and D thresholds for any cell line experiment it was possible to calculate the false positive, specificity and sensitivity rates. Bacterial RNA transcripts containing specific sequence deletions were placed each polyA+ RNA sample. Bacillus subtilis genes/operons were used to estimate the FP rate: lys (LYS, 1612 bp, Acc. No. X17013); spo0B, obg, pheB, pheA (PHE, 3360 bp, Acc. No. M24537), thrC, thrB (THR, 2400 bp, Acc. No. X04603);jojC-birA (DAP, 6540 bp, Acc. No. L38424); trp operon (TRP, 2525 bp, Acc. No. K01391: bp. 1883-4404). The entire sequences of these loci were tiled on the DGCR chip. For the Chrom 21_{—}22 arrays, probes were picked every 30bp from the following regions of each gene/locus used: LYS 328-1344; PHE 2016-3331; THR 247-2231; DAP 1357-3196; TRP 1-2517 using identical probes selection rules as for the rest of the genomic sequences. A polyadenylated transcript corresponding to a smaller portion of each five loci was generated to evaluate the sensitivity of the assay, while the bacterial region outside of the spiked regions was employed in determination of the FP rates. The regions of each gene/locus corresponding to spiked transcripts are: LYS 817-1344; PHE 2852-3331; THR 1221-2231; DAP 1357-2493; TRP 1-1261. The control bacterial transcripts were spiked into human polyA+ preparations before cDNA synthesis procedure at following concentrations (copies/cell): LYS and PHE- 3; THR and DAP-10 and TRP-30, assuming 300,000 different mRNA species in a human cell and size of an average transcript 1300 nt.

False negative (FN) rates for these array experiments were estimated by using the present segments of the spiked bacterial RNA control transcripts, as well as exon sequences determined to be present in the polyA+ RNA samples extracted from each cell line by means of reverse transcriptase-mediated PCR (RT-PCR) amplification assays. A total of 52/99 exon regions were detected as being present in the extracted poly A+ RNA. From these experiments, it was also possible to determine false positive (FP), sensitivity (Sn) and specificity (Sp) values for each cell line for a set of fixed R and D values. Maps of a certain target false positive rate were generated by fixing the maxgap, minrun and D values, then adjusting R over the range 1.1 to 1.5 until the target false positive rate was reached in the bacterial controls. If the target rate was not achieved over the specified range of R the value achieving the closest was used.

For the array interrogating each base in the chromosome 22 DGCR, Table 1A illustrates that at a 5% FP rate a range of 47-65% Sn for the bacterial control sequences and 15-26% for the human exonic RNA sequences. Table 1B provides similar data for the chrom 21_{—}22 array experiments at fixed R and D values. These data highlight the point that use of the bacterial control sequences as controls to evaluate Sn and Sp values may result in a higher sensitivity than the use of human exonic sequences. The differences in the bacterial and human Sn values can be attributed to differences in concentrations existing between the bacterial and human targets, to the differences in the nucleotide composition and sequence of the two types of controls (human and bacterial) in terms of their interaction with competing RNA found in human cells.

TABLE 1
Sensitivity and Specificity Estimates
A. DGCR (22q 11.2)^{1.}

| Cell Lines | BacSp2² | BacSn³ | HumSn⁴ | pct.Pos⁵ | pct.PosUnq⁶ |
|---|---|---|---|---|---|
| A-375 | 0.857 | 0.487 | 0.167 | 21.72 | 14.561 |
| CCRF-CEM | 0.817 | 0.613 | 0.221 | 20.642 | 11.077 |
| COLO 205 | 0.820 | 0.652 | 0.185 | 18.772 | 8.279 |
| FHs 738Lu | 0.775 | 0.473 | 0.261 | 22.872 | 14.499 |
| HepG2 | 0.795 | 0.555 | 0.240 | 23.203 | 15.82 |
| Jurkat | 0.783 | 0.542 | 0.153 | 20.064 | 9.876 |
| NCCIT | 0.804 | 0.545 | 0.162 | 21.664 | 9.584 |
| NIH: | 0.785 | 0.504 | 0.243 | 20.721 | 10.908 |
| OVCAR-3 | | | | | |
| PC3 | 0.792 | 0.559 | 0.161 | 17.35 | 6.765 |
| SK-N-AS | 0.873 | 0.259 | 0.109 | 16.708 | 9.676 |
| U-87 MG | 0.822 | 0.641 | 0.187 | 18.76 | 7.335 |

^{1.}Estimates made at a ˜5% FP rate with the exception of A-375 (FP = 3%)
and SK-N-AS (FP = 1.4%), R values range from 1.17-1.47 (17,18).
^{2.}Bacterial specificity,
^{3.}Bacterial sensitivity.
^{4.}Human Sensitivity
^{5.}Percent positive probes in the entire 360 kb DGCR.
^{6.}Percent positive probes in non-repetitive sequences of the 360 kb DGCR.
For the bacterial controls: the FP rate calculated as proportion of probes
called positive in the regions of the bacterial controls absent in the sample;
the BacSp2 was calculated from the formula TP/(TP + FP), where TP is
the number of positive probes in the present regions of the bacterial
controls and FP- number of positive probes in the deleted regions of the
bacterial control and the BacSn was calculated from TP/(TP + FN) with
FN being the number of negative probes in the present regions of bacterial
controls.
For the human DGCR region: HumSn is a fraction of probes called
positive within the 52 exons or parts of exons corresponding to the known
genes (DGCR6, DGCR2 exons 6-10, DGS-I, DGS-H, DGS-A, SLC25A1
exons 1-4 and Clathrin) and one validated locus RP8 shown to be present
in the human cell lines using RT-PCR. The exact coordinates and
descriptions of the regions used to calculate the HunSn rate could be
found at http://www.netaffx.com/transcriptome.

B. Chromosomes 21-22^{1.}

| Cell Lines | BacSp2 | BacSn | BacFp | pct. Pos | pct. Pos Exn |
|---|---|---|---|---|---|
| A-375 | 0.941 | 0.711 | 0.046 | 0.062 | 0.272 |
| CCRF-CEM | 0.88 | 0.861 | 0.121 | 0.115 | 0.44 |
| COLO 205 | 0.858 | 0.864 | 0.148 | 0.121 | 0.445 |
| FHs 738Lu | 0.874 | 0.735 | 0.117 | 0.094 | 0.341 |
| HepG2 | 0.886 | 0.859 | 0.114 | 0.099 | 0.386 |
| Jurkat | 0.926 | 0.742 | 0.061 | 0.073 | 0.335 |
| NCCIT | 0.904 | 0.787 | 0.088 | 0.086 | 0.341 |
| NIH: OVCAR-3 | 0.86 | 0.817 | 0.139 | 0.107 | 0.433 |
| PC3 | 0.853 | 0.829 | 0.151 | 0.145 | 0.447 |
| SK-N-AS | 0.949 | 0.646 | 0.036 | 0.059 | 0.234 |
| U-87 MG | 0.839 | 0.854 | 0.17 | 0.127 | 0.44 |

^{1.}Thresholds fixed for all cell lines at R = 1.3 and D = 12Q (17). BacFP
rate varies, see footnote to Table 1A.

High Resolution Map of DGCR

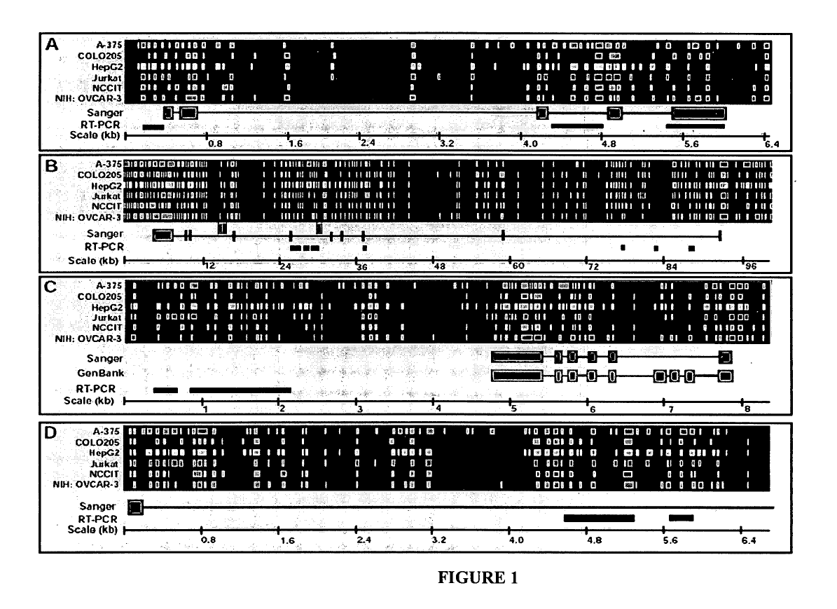

As expected, the maps generated for chromosomes 21 an 22 are highly fragmented because of several reasons, including: the use of a single set of thermodynamic conditions for hybridization, probe-pair specific hybridization properties, relatively sparse spacing of the probe pair cross-hybridization of partially complementary sequences and the need for algorithmic development to predict the structural relationship between two neighboring positive probes. One approach to reduce the fragmentary nature of the maps is to increase the density of the interrogating probes. Transcriptionally active regions of the DGCR (22q11.2) were mapped using oligonucleotide probes spaced every bp for 362,901 bp. Both repetitive (42%) and non-repetitive (58%) sequences were interrogated by this array. The first transcription map for a portion of this region was constructed by Gong, et al. (Gong, W., et al. Human Mol Genet 5, 789 (1996); Gong, W, et al. Human Mol Genet 6, 267 (1997)). Thirteen well-characterized genes (99 exons) and 2 pseudogenes have been mapped to the DGCR. A high-resolution map describing the locations of both annotated exonic sequences and the array-based detected transcriptionally active regions has been developed and four of the annotated genes from this region are depicted in FIG. 1. The use of overlapping probe pairs allowed for the construction of contigs within this region and assisted in the defragmentation of the map. The formation of contigs for this map allowed us to lower the estimated FP rate for each of the 11 cell lines to 3-5% with sensitivities ranging from 15-25% based on the human control sequences (Table 1A). Similar to that observed with the maps of chromosomes 21 and 22 most of the detected transcripts (59.4-65.9%) are located away from the annotated exonic and EST sequences (Table 2B).

TABLE 2
Proportion of Genome Transcribed
A. Chromosomes 21-22^{1.}

| | Pos. Probes | Pos. Probes |
|---|---|---|
| Cell Lines | Overall | In Exons |
| 1 of 11 | 268,466 (26.5%) | 17,924 (67.6%) |
| 5 of 11 | 98,231 (9.7%) | 10,903 (41.1%) |

^{1.}(1,011,768 probes, 26,516 query exons as annotated in the known
mRNAs such as RefSeqs, Sanger hand-curated and GenBank mRNAs,
ESTs not included as part of expressed portion of genome).

B. DGCR (22q 11.2)^{1.}

| | | Pos NR | Pos NR | Pos NR |
|---|---|---|---|---|
| Cell Lines | FP^{2.} | Bases | Expr Bases^{3.} | Non-Expr Bases |
| 1 of 11 | 3% | 50,885 (23.9%) | 17,421 (34.2%) | 33,464 (65.8%) |
| | 5% | 63,908 (30.0%) | 21,788 (34.1%) | 42,120 (65.9%) |
| 5 of 11 | 3% | 11,623 (5.5%) | 4,724 (40.6%) | 6,899 (59.4%) |
| | 5% | 20,097 (9.7%) | 7,477 (37.2%) | 12,620 (62.8%) |

^{1.}The values are calculated on the basis of 213,009 probes interrogating
non-repetitive bases of which 61,842 probes are located within annotated
expressed regions of the DGCR;
^{2.}The target FP rate for each individual cell line.
^{3.}Refers to the databases mentioned in Table2A plus all the ESTs mapping
to this region.

By using a combination of a higher resolution analysis array and by selecting the most mature subfraction of RNA transcripts specifically transported from the nucleus, additional information about the annotated portion of the transcriptome can also be revealed. For example, DiGeorge Critical Region gene 6 (DGCR6) is the first gene in the DGCR (Demczuk, S., et al. Human Mol Genet 5, 633 (1996)). Using the DGCR array, analysis of the transcriptionally activity of this annotated region provides novel information concerning both exon and intron structures of this gene. FIG. 1A illustrates of the current annotated structure for DGCR6 created using the Sanger-hand curated database (http://www.sanger.ac.uk/HGP/Chr22). The map produced by the DCGR array using a 5% FP error estimate indicates that exons 1 and 5 may be longer than previously represented and that there is evidence for transcriptional activity within intron 3. RT-PCR analysis and subsequent cloning/sequencing of the PCR products confirmed the array data and resulted in the identification of both the canonical and alternative forms of DGCR6 exons 1 and 5 as well as transcription activity within intron 3. Interestingly, recent studies by Edelmann, et al support these data for an extension of length of exon 1 and an alternate splice form for DGCR6 that does not remove intron 3 (26. Edelmann, L., et al. Genome Research 11, 208 (2001)).

Similar alterations could be made to the annotations of three other regions of the chromosome 22 DGCR (FIG. 1B-D). The ten exon DGCR2 gene (FIG. 1B) contains two non-coding genes within introns 3 (DGSyndD) and 5 (DGSyndE) (22). RT-PCR analysis and subsequent sequencing of the transcripts in intron 5 revealed an extended version of DGSyndE as well as transcripts 5′ to this gene. Additional limited RT-PCR analysis provided confirmatory evidence for the presence of other transcripts in the DGCR2 locus (FIG. 1B). Similarly, novel transcripts have been observed and confirmed in the intron1 of DGCR5 (FIG. 1D) and the 5′ region from the highly expressed SCL25A gene. Additional supportive evidence for the array-detected transcripts observed in the DGCR comes from ESTs mapped to this region. Thus, these maps have not only been useful in estimating the overall fraction of the human genome that is transcribed but also as a guide for directing further biochemical and molecular efforts to isolate novel transcripts. High resolution maps for the entire sequences of the DGCR and the non-repeat sequences of chromosomes 21 and 22 are also available.

Transcriptionally Active Loci of Chromosomes 21 and 22

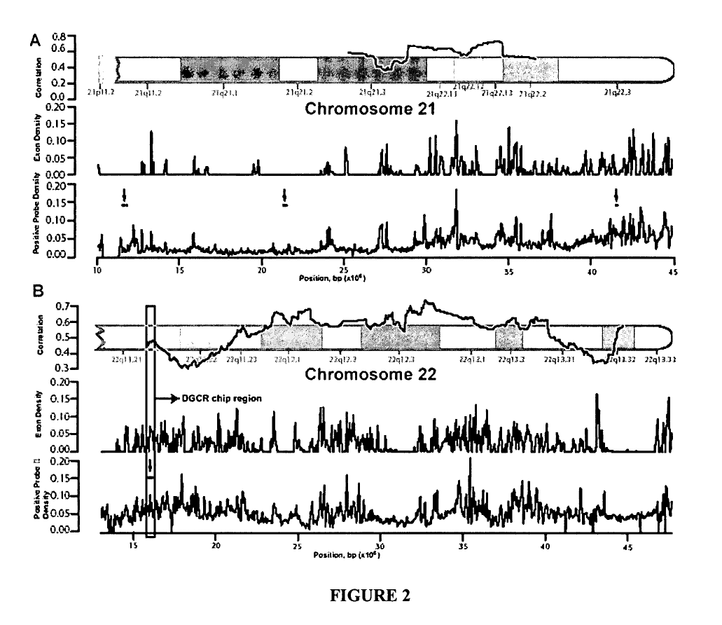

Chromosomes 21 and 22 have at least 225 and 545 well-characterized and predicted genes, respectively. Of these approximately 127 and 247 are well characterized, “known genes” (Dunham, I, et al. Nature 402, 489 (1999); Hattori, M., et al. Nature 405, 311 (2000)). These well-characterized genes contain approximately 1430 and 3134 exons on chromosomes 21 and 22, respectively (Best in genome alignments of Refseq, cmma and Sanger sequences have been used to produce a list of the union of exon sets.). FIG. 2 provides an overview of the previously identified and array predicted transcription activity on chromosomes 21 and 22. By dividing the non-repeat genome sequences of chromosomes 21 and 22 (˜35 Mb) into 57 Kb increments (average gene length on chromosome 21) (Hattori, M., et al. Nature 405, 311 (2000)), a total of 620 gene-sized loci can be created across both chromosomes. Given that the average distance between each interrogating probe pair is 30 bp, the positive probe and exon densities (It was calculated that the fraction of positive probe pairs as the number of probe pairs defined as positive using R=1.5 and D=12Q in at least 8 of 11 cell lines divided by the number of interrogating probe pairs, in non-overlapping 57Kb windows for both chromosomes 21 and 22) for each loci is plotted and can be compared. The correlation between the exon and positive probe densities demonstrated a non-random relationship over the majority of the lengths of both chromosome sequences. Of the 1,011,768 probe pairs that interrogate approximately 35,000,000 non-repetitive bp of both chromosomes, 26,516 (2.6%) probe pairs are located within the 4,564 annotated exons of well-characterized genes. Totals of 69.8% and 40.7% of these annotation-focused probes detect RNA transcript, in at least 1 or 5 of 11 cell lines, respectively (Table 2A). The percent of the overall positive probes detected was 34.8% and 9.6% of the 1,011,768 probes in 1 or 5 of 11 cell lines, respectively. This indicates that 94% and 88% of the probes detecting transcripts are located outside annotated exons in 1 or 5 of 11 cell lines, respectively. Approximately, 50% of these positive probes are located >300 bp distant from the nearest annotated exon. This is reflected in the close correlation between the positive exon and probe densities.

Verification of Mapping Results

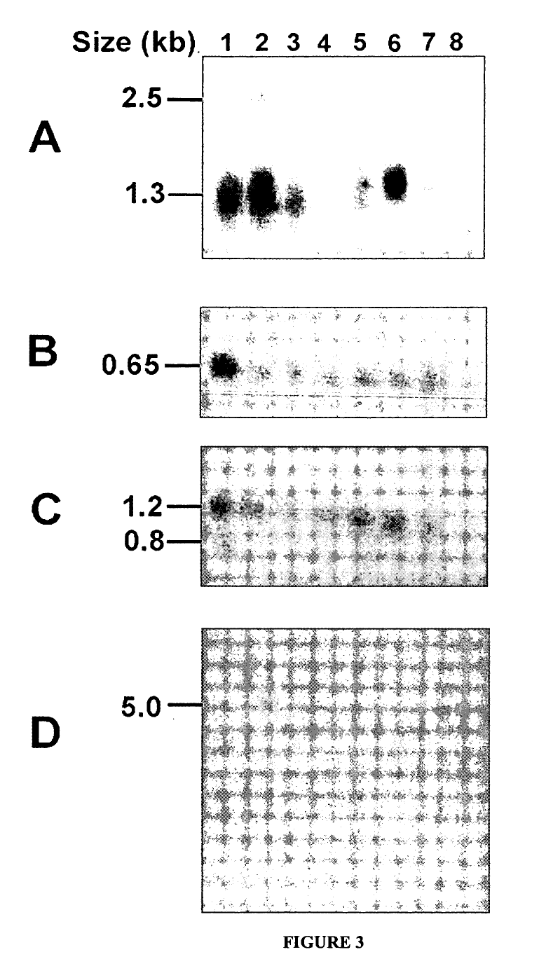

Errors in detecting a complementary RNA target at the probe pair level were estimated by measuring FP and FN rates using spiked and endogenous RNA control sequences. Determining what are the structures of the RNAs detected by both the DGCR and 21_{—}22 arrays involved the use of three different experimental approaches. Fourteen individual, array-predicted transcription sites located within 14 dispersed gene-sized loci on chromosomes 21 and 22 distant from annotated exons (FIG. 2) were selected as sites for independent verification and analyses (Table 3). Reverse transcriptase- mediated PCR (RT-PCR) reactions were carried out using primers derived from the sequences of the positive probe regions detected by the arrays and the cytosolic poly A+ RNA as template (RT-PCR procedure was carried out using the C. therm. Polymerase One-Step RT-PCR System (Roche). The RT-PCR procedure used 10-50 ng of cytosolic polyA+ RNA from each of specified cell lines following the manufacturer's instructions. At least 40 cycles of amplification were required to see the products. PCR products were cloned in pCR4-TOPO vector (Invitrogen) and sequence of the products determined). Predicted PCR products ranging in size from approximately 178 to 1036 bp were cloned and sequenced from 12 of these loci. Nucleotide sequences for five of these PCR products were observed to be unique to chromosomes 21 or 22. The remaining analyzed regions have homologue copies present on other chromosomes. RNA products transcribed from each homologue site were distinguishable from the transcripts originating from the analyzed chromosomes. In all cases, at least a portion of the RNA transcripts detected emanated from the chromosome 21 or 22 homologue and was co-linear with the published genome human sequence. Additional confidence in the array-predicted results was obtained by the generation PCR products of the expected lengths and sequence for 9 of the 12 loci from cDNA libraries created from cytoplasmic RNA of HepG2 and NIH:OVCAR-3 cell lines. Of the 9 loci for which a PCR product was obtained from the cDNA libraries, partial or full-length clones are being isolated and sequenced. Finally, Northern hybridization experiments were also conducted using poly A+ RNA from 7 of the 11 cell lines as targets (A-375, CCRF-CEM, COLO 205, FHs 738Lu, HepG2, Jurkat, NIH:OVCAR-3) (Northern blot experiments were performed using standard techniques (Sambrook J., Fritsch E. F, and Maniatis, T. Molecular Cloning. A laboratory manual, Ed.2. Cold Spring Harbor Laboratory Press, Cold Spring Harbor, N.Y.). 3-5 μg of cytosolic polyA⁺ RNA from each of specified cell lines was loaded on the gel. DNA probes were labeled with [α-³²P]-dCTP (Amersham) using random hexamer labeling kit (Roche). Filters were hybridized in 0.5M sodium phosphate buffer pH. 7.2, 1% Bovine Serum Albumin, 7% SDS at 65° C. overnight. After hybridization, filters were successively washed at 65° C. in 2×SSC, 0.1% SDS; 1×SSC, 0.1% SDS and 0.3×SSC, 0.1% SDS, 15 min each wash and exposed to X-ray film for 3 weeks.). Each of the cloned and sequenced RT-PCR products was labeled and used as a probe for these hybridization experiments. Four of the twelve loci from chromosomes 21 and 22 contained identifiable full-length transcripts in at least one of the seven cell lines tested (FIG. 3). One of the loci (Chr21-9) hybridized to transcripts of heterogeneous size ranging from 1-10 kb (data not shown). Using Northern hybridization analysis, an additional 4 other loci were analyzed from the DGCR2 region. The hybridization results indicated an additional two more heterogeneous group of transcripts. Thus, by Northern hybridization analyses, seven of 16 loci yielded detectable RNA transcripts with several loci characterized by multiple transcripts of distinct or indistinct size ranging in size from 0.6 to 10 kb.

In summary, the RT-PCR and sequence analyses of the cytosolic poly A+ RNA samples and cDNA libraries indicated that 12/14 loci predicted by the array experiments to be sites of novel transcripts were transcribed. In addition, experiments aimed at directly detecting and determining the size of the full lengths of these RNAs using Northern hybridization experiments revealed that they were typically that of mature, processed RNAs. Interestingly, the sequences collected from the RT-PCR amplicons approached the full length or a considerable portion of the size indicated by some the Northern hybridization products. Sequence analysis of these amplicon products revealed little coding capacity present in these characterized portions of the novel transcripts. Finally, the filter-based hybridization experiments strongly suggest that the observed novel RNAs are present at very low copy number per cell, providing some explanation as to why these transcripts have not previously been observed. While the absence of detectable RNAs by Northern hybridization for the 7 loci is also consistent with very low copy number representation for these transcripts, it is important to emphasize that these transcripts were detected as part of the cDNA libraries that were examined using primer pairs whose sequences were suggested from the array data.

TABLE 3
RT-PCR Verification of Array Detected Transcripts¹

| Number | Nam | PCR start² | PCR end² | PCR length | Library³ | Other Chr.⁴ |
|---|---|---|---|---|---|---|
| 1 | Chr22 DGCR-1-1 | 11463 | 11753 | 194 | N/T | Dup. on 22 |
| | Chr22 DGCR-1-2 | 15486 | 15973 | 487 | N/D | Dup. on 22 |
| | Chr22 DGCR-1-3 | 16627 | 17211 | 584 | N/T | Dup. on 22 |
| 2 | Chr22 DGCR-2-1 | 164261 | 164831 | 570 | N/D | Unique on 22 |
| | Chr22 DGCR-2-2 | 162186 | 163222 | 1036 | N/D | Unique on 22 |
| | Chr22 DGCR-2-3 | 165841 | 166370 | 529 | N/D | Unique on 22 |
| 3 | Chr22 DGCR-3-1 | 276148 | 276490 | 342 | NIH: OVCAR-3 | Unique on 22 |
| | Chr22 DGCR-3-2 | 276727 | 278050 | 1323 | NIH: OVCAR-3 | Unique on 22 |
| | | | | | and HepG2 | |
| 4 | Chr22 DGCR-4-1 | 80161 | 80863 | 702 | N/D | Dup. on 22 |
| | Chr22 DGCR-4-2 | 81278 | 81538 | 260 | N/D | Dup. on 22 |
| 5 | Chr21-1 | 41484371 | 41484656 | 285 | NIH: OVCAR-3 | Unique on 21 |
| 6 | Chr21-2* | 41515490 | 41516422 | 932 | N/T | N/T |
| 7 | Chr21-3* | 41532516 | 41533480 | 964 | N/T | N/T |
| 8 | Chr21-4 | 41539789 | 41540256 | 467 | N/D | Unique on 21 |
| 9 | Chr21-5-1 | 21332449 | 21332920 | 471 | N/D | Chr.11, 18 |
| | Chr21-5-2 | 21333394 | 21334037 | 643 | HepG2 | Chr.11, 18 |
| | Chr21-5-3 | 21334196 | 21334355 | 159 | HepG2 | Chr.11, 18 |
| 10 | Chr21-6 | 21320916 | 21321771 | 855 | HepG2 | Chr.5, 14 |
| 11 | Chr21-7 | 21471231 | 21471568 | 337 | HepG2 | Unique |
| 12 | Chr21-8 | 11773874 | 11774085 | 211 | HepG2 | Chr. 13, 17, 18 |
| 13 | Chr21-9 | 11604183 | 11604877 | 694 | HepG2 | Dup. on 21, Chr.18 |
| 14 | Chr21-10 | 11538194 | 11538927 | 733 | HepG2 | Dup. on 21, Chr.2 |

¹.Several PCR primer pairs were designed for each of the 14 loci in the regions called positive by the chip. Primers were typically picked at or near positive probes or contigs (in case of the DGCR region) with a distance between forward and reverse primer on the order of 200-500 bp. Typically, 3 to 15 primer pairs designed for each loci. For the DGCR region (Chr22 DGCR), the 5% FP maps were used for primer selection, while for the Chromosome 21 regions (Chr21), 1of 11 map with R=1.3 and D=12 was used. For some loci, more then one region was validated by RT-PCR. Start and end of each validated region is shown either in the coordinates of the sequence of the DGCR region tiled on the chip for the Chr22 DGCR loci or in the coordinates of the October 2000 freeze of the Golden Path sequence for the Chr21regions. The cDNA libraries from HepG2 and NIH: OVCAR-3 were used to detect clones which contain RT-PCR products identical to that isolated from the poly A⁺ RNAs of these cells. Locations on other chromosomes which have sequences similar to that identified in the RT-PCT products as shown by the BLAT search (http://genome-test.cse.ucsc.edu/cgi-bin/hgBlat). In all cases in which a homologue was identified elsewhere on the genome, the RT-PCR products specific to sites interrogated on chromosomes 21 and 22 were observed because of chromosome 21 or 22 loci-specific SNPs. * No RT-PCR products were detected for these loci. N/T-not tested; N/D-detected.

V. Example—Methods and Compositions for Determining the Biological Significance of Transcriptional Activity

It has been previously demonstrated that transcriptional activity is not limited to the annotated and predicted exons of human chromosomes 21 and 22 (Kapranov et al. Science 2002;296: 916-919) using oligonucleotide (25-mers) microarrays with complete coverage of the known genomic sequences and an average spacing of 10 bases between the probes. It has been shown, for example, that there may be as much as an order of magnitude more transcriptional activity than can be accounted for by the annotations available for these chromosomes. For a detailed description see, for example, U.S. patent application Ser. No. 10/316,518 incorporated herein by reference.

The functional/biological significance of these transcripts were investigated using the expression profiling methods of the present invention. NCCIT (teratocarcinoma, ATCC no. CRL-2073), a developmentally pluripotent human germ cell tumor-derived cell line was chosen as a model biological sample in order to assess the functional significance of these transcripts. One of skill in the art would appreciate that the methods of the present invention are not necessarily restricted to the NCCIT cell line or a developmentally pluripotent human germ cell tumor-derived cell line. Cell lines such as the Jurkat (acute T cell leukemia; ATCC TIB-152) or HL-60 (human leukemia) may also be employed for the methods of the invention. NCCIT cells display some properties of embryonic stem cells, including their ability to undergo retinoid-induced differentiation into keratin and neurofilament-positive somatic cells. These differentiated cells are also capable of extra cellular matrix (ECM) deposition. Additionally, NCCIT cells yield high quantities of polyA+ RNA and also facilitate preferential enrichment of cytoplasmic RNA, enhancing the amount of mature transcripts being analyzed.

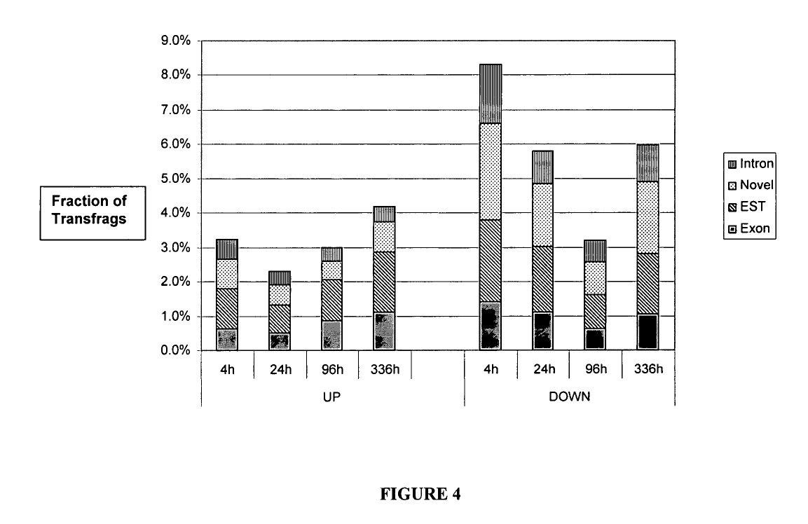

Empirical transcriptional maps of NCCIT during various stages of differentiation were generated from polyA+ enriched cytosolic RNA using the chromosome 21 and 22 genome tiling arrays. NCCIT cells were stimulated with 10 μM retinoic acid (RA) and polyA+ RNA was extracted at 4, 24, 96 and 336 hours of stimulation (FIG. 4). One of skill in the art would appreciate that the methods of the invention are not restricted to Retinoic Acid being the morphogenic/differentiating stimulant for NCCIT cells. Other inducers such as phorbol esters, for example, may also be employed. The cDNA assay employed does not allow for direct determination of strandedness, nevertheless regions proximal to, internal or 3′ of transcription factor binding sites (TFBS) were chosen to represent potential antisense transcripts. Coordination in expression levels between exons of well-characterized transcripts and these putative antisense regions was monitored by probes overlapping sense/antisense transcript pairs identified from public databases, estimating the signal for each region and determining a correlation coefficient for sense/antisense pairs over the time points of the RA treatment. For each transcript partner of a sense-antisense pair, as well as for each time point, the median fold change with respect to a control for all probes interrogating the transcript was evaluated.

The correlations on chromosome 22 exhibit a significant increase in both location and spread over what would be expected at random, suggesting the existence of a sub-population of genes where the sense and putative antisense transcripts are positively correlated. For chromosome 21, 18 of the correlations are greater than 0.9, whereas only 9 would be expected at random; the corresponding figures for chromosome 22 and 93, 0.93 and 33. The thresholds have been selected to obtain a false positive rate of 5%. The positive correlation is consistent with various other interpretations, including that of ectopic transcription from the sense strand, or as-yet undiscovered alternative splicing or polyadenylation, and is also consistent with the presence of antisense transcription in an unexpected positive regulatory role for corresponding sense transcripts.

In some ways, the positive correlation is unexpected, since the role of antisense transcripts has sometimes been associated with the negative regulation of sense transcript expression. Such anticipated reciprocal regulation is not observed on a global chromosome level. Of the approximately 10% (21/214) of the sense/antisense gene pairs that respond during the time course of retinoic acid stimulation, only a few show this anticipated reciprocal regulation. The positive coordinated expression of sense and antisense gene pairs point to a supportive function for the expression of the responding genes and also shows that such antisense transcription in not a random and unguided response. Another interesting observation stemming from the results is that these polyadenylated sense and antisense transcripts are destined for the cytosol, from which they were isolated, suggesting that the possible supportive functions for antisense transcription occur in a separate cellular compartment, in contrast to negative regulatory antisense RNAs, which are postulated to have a nuclear location (Carmichael, Nature Biotechnology 21, 371-2, April 2003). All these observations are consistent with a functional role for a large subset of the earlier reported novel transcripts.

When regions whose expression was changed by 2 fold or more were subdivided into different annotation categories, it can be observed that the fraction that maps to Novel/Intronic regions represents a major subpopulation of varying transcriptional fragments (transfrags), especially the down-regulated fraction, where that represents up to 50% of all down-regulated transfrags (FIG. 4). Expression pattern of novel transfrags changes in a manner similar to that of the known genes.

Overall, these results suggest that the expression levels of previously uncharacterized transcripts demonstrate highly reproducible regulated responses during various stages of differentiation, underscoring their potential as functionally significant transcriptional entities. Understanding the functional significance of the hidden transcriptome has the potential to tremendously increase the repertoire of available genomic targets for drug discovery and development purposes.

Conclusion

These examples show that the exemplary embodiments of the methods of the invention are powerful tools for exploring the transcriptome. For example, in this example, cytoplasmic poly A+ RNA obtained from 11 developmentally diverse cell lines indicated that there may be as much as 9 fold greater sites of transcription of mature RNA that is transported into the cytoplasm than can be accounted for by the previous annotation of the sequence of the human genome.

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method of transcriptional profiling comprising:
subjecting a biological sample to an exogenous stimulation;
measuring transcriptional activity of the biological sample at a first differentiation stage;
measuring transcriptional activity of the biological sample at a second differentiation stage; and
comparing the transcriptional activities from the first and second differentiated stages in at least 5 Mbases, 50 Mbases or 100 Mbases of the genome to obtain a transcription profile.

2. The method of claim 1 wherein the measuring comprises obtaining polyA+ enriched cytosolic RNA and hybridizing the RNA to high density oligonucleotide probe arrays.

3. The method of claim 2 wherein the oligonucleotide probe array contains at least 100,000 oligonucleotide probes, each targeting a transcript sequence from a different region of a genome.

4. The method of claim 3 wherein the oligonucleotide probe array contains at least 500,000 oligonucleotide probes, each targeting a transcript sequence from a different region of a genome.

5. The method of claim 4 wherein the oligonucleotide probe array contains at least 800,000 oligonucleotide probes, each targeting a transcript sequence from a different region of a genome.

6. The method of claim 5 wherein oligonucleotide array further comprises mismatch (MM) probes, wherein each of the mismatch probes is different from a perfect match (PM) probe in one base.

7. The method of claim 6 wherein the mismatch probe is different from the perfect match probe in a middle position.

8. The method of claim 7 wherein the biological sample is responsive to an exogenous stimulation.

9. The method of claim 8 wherein the biological sample is a developmentally pluripotent human germ cell tumor-derived cell line.
